(19) United States
(12) Patent Application Publication
(10) Pub. No.: US 2026/0286314 A1
(43) Pub. Date: Sep. 24, 2026

(54) NF2-ASSOCIATED MENINGIOMA MODEL

(71) Applicant: THE RESEARCH INSTITUTE AT NATIONWIDE CHILDREN'S HOSPITAL, Columbus, OH (US)

(72) Inventor: Long-Sheng CHANG, HILLIARD, OH (US)

(21) Appl. No.: 19/087,110

(22) Filed: Mar. 21, 2025

(51) Int. Cl. C12N 5/09; A01K 67/0271; A61K 31/519; A61K 31/675; A61P 35/00

(52) U.S. Cl. CPC C12N 5/0693; A01K 67/0271; A61K 31/519; A61K 31/675; A61P 35/00; A01K 2207/12; A01K 2227/105; A01K 2267/0331; C12N 2510/04

(57) ABSTRACT

A method of generating a tumor cell line is described. The method includes transfecting meningioma cells obtained from a benign tumor with telomeres reverse transcriptase to provide telomerase-immortalized meningioma cells, and passaging the telomerase-immortalized meningioma cells in growth medium together with untransformed meningioma cells until the untransformed cells undergo senescence to provide a tumor cell line comprising telomerase-immortalized meningioma cells. A tumor cell line including telomerase-immortalized meningioma cells obtained from a benign tumor is also described, as well as an animal model of NF2-deficient meningioma that includes a mammal implanted with telomerase-immortalized, luciferase-expressing meningioma cells obtained from a benign tumor.

### GOVERNMENT FUNDING

This invention was made with government support under W81XWH-18-1-0547 awarded by the Department of Defense. The government has certain rights in the invention.

### BACKGROUND

Presently, an FDA-approved targeted therapy is not available for meningiomas, tumors originating from meningothelial cells of the arachnoidal layer lining the brain. The majority (~80%) of meningiomas are benign (WHO grade I), and the remaining are atypical (grade II) and anaplastic (grade III). These tumors cause significant morbidity, including seizure, cranial nerve palsy, and brainstem compression, which may lead to paralysis, aspiration pneumonia, and death. Surgical resection and radiation are current treatment options. However, complete resection of tumors is often difficult, especially for those located along the skull base.

Meningiomas can occur sporadically in patients with neurofibromatosis type 2-related schwannomatosis (NF2 or NF2-SWN), an extremely debilitating tumor suppressor syndrome which predisposes affected individuals to the development of multiple nervous system tumors, including multiple meningiomas. NF2 is caused by mutations that inactivate the NF2/merlin gene. Studies have shown that merlin-deficient meningioma cells exhibit cytoskeletal and cell contact defects, altered cell morphology and growth properties, and susceptibility to senescence. In addition, NF2 activation in arachnoidal cells or meningeal precursor cells in mice leads to meningioma formation. While studies of these genetically engineered mouse models confirm the merlin's tumor suppressor role, they have not been useful for therapeutic evaluation due to the small size and location of the tumors.

A number of animal models for studying meningioma are known [see Anderson et al., J Transl Med., 21(1): 764 (2023)]. Over the last 40 years of research, about 70% of meningioma studies have used established, commercially available cell line models. However, an NF2-associated meningioma model using benign tumor cells commonly found in patients with NF2 is not available.

### SUMMARY OF THE INVENTION

Previously the inventors showed that the Ben-Men-1 cell line, which was established from a grade-I meningioma isolated from a sporadic patient is completely NF2-deficient [Burns et al., Cancer Res. 73(2): 792-803 (2013)]. The inventors also generated luciferase-expressing Ben-Men-1 cells. By stereotactically implanting luciferase-expressing Ben-Men-1-LucB cells in the skull base of immunodeficient mice and monitoring tumor growth over time by bioluminescence imaging (BLI), the inventors established a quantifiable orthotopic NF2-deficient meningioma model. Using this model, they have identified several potential targeted drugs or drug combinations for these tumors. In particular, they found that the multi-tyrosine kinase inhibitor brigatinib causes tumor shrinkage in Ben-Men-1-LucB xenografts.

To establish a meningioma model that is more representative of NF2, the inventors have generated a telomerase-immortalized meningioma cell line from a grade-I tumor obtained from an NF2 patient. This NF2-associated meningioma cell line, designated as AG-NF2-Men-1, retained many characteristics of the original tumor. Morphologically it looks fibroblastic and is slow growing in culture, relative to malignant cancer cell lines. Also, AG-NF2-Men-1 cells expressed several meningioma markers, but not the NF2-merlin protein, indicating that they are NF2-null. Like Ben-Men-1 cells, AG-NF2-Men-1 cells expressed several receptor-tyrosine kinases (RTKs) such as EGFR, ErbB3, and IGF-1R, frequently activated in NF2-deficient tumors, and responded to their cognate ligands. In addition, they isolated luciferase-expressing AG-NF2-Men-1 derivatives and implanted them in the skull case or subcutaneously in the back of immune-deficient mice. Interestingly, the luciferase-expressing AG-NF2-Men-Luc2 cells readily grow orthotopically but not at the subcutaneous location, suggesting the tumor microenvironment plays an important role in supporting the growth of NF2-associated meningiomas. Using this newly established NF2-associated meningioma model, the inventors also found that brigatinib effectively blocked AG-NF2-Men-Luc2 meningioma growth with some tumor shrinkage. In summary, the inventors established a quantifiable orthotopic NF-2 associated meningioma model that can enhance NF2 translational research, among other things.

### BRIEF DESCRIPTIONS OF THE DRAWINGS

The present invention may be more readily understood by reference to the following figures, wherein:

FIGS. 1A-1G provides images showing the morphological and immunological characterization of the AG-NF2-Men tumor and established cell line. (A-B) Hematoxylin and eosin (H&E) staining of tumor sections of the AG-NF2-Men meningioma from an NF2-SWN patient show typical whorling architecture and psammoma bodies. (C) IHC (Immunohistochemistry) analysis of AG-NF2-Men tumor sections showed membrane and cytoplasmic expression of EMA (Epithelial Membrane Antigen), commonly found in meningioma cells. (D) Immunofluorescence staining detected vimentin expression (green) in the cytoplasm of AG-NF2-Men cells. DAPI (4′,6-diamidino-2-phenylindole) stained the nuclei in blue. (E-F) Phase contrast micrographs of the AG-NF2-Men primary culture (E) and immortalized cells (F). Like the primary culture, immortalized AG-NF2-Men cells exhibit a spider web-like growth pattern. (G) Western blot analysis confirmed that like the Ben-Men-1 cell line established from a sporadic grade 1 meningioma, AG-NF 2-Men cells did not express detectable merlin protein, while normal meningeal cells (MC) and HeLa cervical carcinoma cells readily expressed merlin. Also, the merlin protein was not detected in primary cultures of four NF2-SWN related meningiomas (Men-1 to 4). In addition, desmoplakin (DSP), a desmosomal component often expressed by meningioma cells, was detected in AG-NF2-Men cells, Ben-Men-1 cells, MC, and primary meningioma cells.

FIGS. 2A-2D provide graphs and tables showing immortalized AG-NF2-Men cells exhibit robust telomerase activity. (A) Real-time PCR (Polymerase Chain Reaction) amplification curves of the telomerase-specific amplicons from primary AG-NF2-Men cells compared to hTERT (human telomerase reverse transcriptase)-immortalized AG-NF2-Men and Ben-Men-1 cells. The manufacturer-supplied lysates from telomerase-expressing cells before and after heat-inactivation served as positive and negative assay controls, respectively. A no-template control (H₂O) was used as an additional negative control. The horizontal red line indicates the threshold at which the Ct (threshold cycle) values were measured. (B-C) Melt Curve (B) and Melt Peak (C) analysis of the telomerase-specific amplicons at the end of the PCR analysis. (D) Tables summarizing the mean Ct and melt temperatures of the amplicons, and the estimated fold-increase in telomerase activity in immortalized AG-NF2-Men and Ben-Men-1 cells versus primary AG-NF2-Men cells. (E) An agarose gel showing that high levels of the telomerase-specific amplicons are only present in hTERT-immortalized AG-NF2-Men and Ben-Men-1 cells.

FIGS. 3A-3C provide graphs showing the brigatinib+INK128 combination exhibited synergistic antiproliferative activity in AG-NF2-Men cells. (A) Dose-response curves from proliferation assays of AG-NF2-Men cells treated for 3 days with serial dilutions of each indicated drug established a mean IC₅₀ (50% inhibitory concentration) of 1,100 nM for brigatinib and 20 nM for INK 128. (B) AG-NF 2-Men cells were treated for up to 14 days with DMSO (dimethyl sulfoxide, as the vehicle control) or 1X IC₅₀ of brigatinib or INK128 alone or in combination. Cells were harvested in biological duplicates and counted by hemocytometer. (C) AG-NF2-Men (left) and Ben-Men-1 (right) cells were treated in a combinatorial array with brigatinib and INK128, and cell proliferation was measured relative to DMSO control cells designated 100%. Cell proliferation arrays are shown above and Loewe additivity synergy score matrices are depicted below. Blue shading denotes drug antagonism while red shading indicates enhanced growth inhibition by the brigatinib+INK128 combination. Loewe scores≤−0.1 signify synergistic drug interaction. For line graphs, solid symbols and error bars for both graphs represent the mean +SD (standard deviation) from two independent experiments. The open symbols show the individual average IC50 values (A) and cell numbers (B) from each independent experiment.

FIGS. 4A-4D provide images showing the brigatinib+INK128 combination elicits a superior and more durable suppression of AKT (also known as protein kinase B [PKB] and 4EBP1 (eukaryotic translation initiation 4E-binding protein) phosphorylation, compared to the individual drugs. (A) AG-NF 2-Men cells were treated for 1 and 3 days with DMSO vehicle or 1X IC₅₀ dose of brigatinib (1.1μM) or INK128 (20 nM) as single agents or in combination. Treated cell lysates were subjected to Western blot analysis for total and phosphorylated AKT and the mTORC1 (mammalian target of rapamycin complex 1) target and protein translation repressor 4EBP1. GAPDH (Glyceraldehyde-3-phosphate dehydrogenase) serves as the loading control. (B-D) AG-NF 2-Men cells were serum-starved for 24h to induce quiescence and then treated with brigatinib, INK128, or brigatinib+INK128 in serum-free medium for 2h, followed by growth stimulation for 5 min with 50 ng/mL EGF (Epidermal Growth Factor) (B), Hrg (Heregulin) (C), or IGF-1 (Insulin-like Growth Factor-1) (D). Lysates from these cells were analyzed by Western blotting for ligand-mediated phosphorylation of their cognate RTKs (Receptor Tyrosine Kinases) and downstream phosphorylation and activation of AKT/PRAS 40 (Proline-Rich AKT Substrate of 40 kDa) and ERK 1/2 (Extracellular signal-Regulated Kinases 1 and 2).

FIGS. 5A-5D provide graphs showing AG-NF2-Men cells treated with the brigatinib+INK128 combination exhibit profoundly altered gene expression programs associated with growth suppression, including reduced expression of YAP (Yes-associated protein) target genes. (A) Principal component analysis demonstrating that the biological triplicates from each treatment group cluster together. (B) Venn diagram showing that the brigatinib+INK128 combination-treated AG-NF2-Men cells exhibited 5493 significant unique DEGs (differentially expressed genes) [Padj≤0.01; Abs(Log2 FC)≥1], while single agents only elicited a small number of DEGs. (C) Tables showing a substantially larger number of upregulated and downregulated DEGs of all types (upper table) and of protein-coding genes (lower table) in the combination-treated cells, compared to individual drug treatments. (D) Volcano plots showing DEGs in AG-NF2-Men cells treated with brigatinib (left panel), INK128 (middle panel), and the brigatinib+INK128 combination (right panel), relative to the DMSO-treated controls. Downregulated genes are colored blue and upregulated genes are red, with the top 50 DEGs labeled. The YAP target genes ANKRD1 (Ankyrin Repeat Domain 1), CTGF (Connective Tissue Growth Factor), CPA4 (Carboxypeptidase A4), and CYR61 (Cysteine-rich angiogenic inducer 61) are circled.

FIGS. 6A & 6B provide graphs showing AG-NF2-Men-Luc2 cells did not grow when implanted subcutaneously but readily established intracranial xenografts when injected into the skull base of NSG mice. (A) AG-NF2-Men-Luc2 cells were injected subcutaneously in the back of NSG mice, followed by BLI to monitor tumor growth. The tumor-emitted luminescence was captured every two weeks and the average relative luciferase units (RLU) of tumor-emitted BL signal for the entire group (n=5) was calculated and denoted as % of total flux relative to that of the first scan at two weeks after cell injection, designated as one (100%). (B) AG-NF2-Men-Luc2 cells were stereotactically injected into the skull base of NSG mice and tumor-emitted luminescence captured every week. The mean RLU of tumor-emitted BL signal for the entire group was calculated and relative to the first scan at one week after engraftment (designated as one). Graphs are plotted as mean ±SE (standard error).

FIGS. 7A & 7B provide graphs showing the brigatinib+INK128 combination effectively shrank intracranial AG-NF2-Men-Luc2 meningioma xenografts. (A) Mice with established meningioma xenografts were treated with vehicle, brigatinib, INK128, or the brigatinib+INK128 combination by oral gavage (n =5/group) and tumor growth was monitored by BLI. The relative tumor-emitted BL signals were quantified and denoted as % of total flux after treatment relative to the total flux prior to treatment designated as one (100%). (B) To assess potential tumor regrowth, we stopped treating mice in the INK128 or combination treatment group for 8 weeks and continued monitoring tumor growth by BLI for another 6 weeks. To examine the effects of re-treatment, we re-treated mice and continued BLI monitoring for 6 more weeks. The data are shown as mean ±SE.

### DETAILED DESCRIPTION OF THE INVENTION

The present invention provides a method of generating a benign tumor cell line. The method includes transfecting meningioma cells obtained from a benign (WHO grade I) tumor with a telomerase reverse transcriptase-expressing unit and passaging the telomerase immortalized meningioma cells in growth medium together with untransformed meningioma cells until the untransformed cells undergo senescence to provide a tumor cell line comprising telomerase-immortalized meningioma cells. A tumor cell line including telomerase-immortalized meningioma cells obtained from a benign tumor is also provided, as well as an animal model of NF2-deficient meningioma that includes a mammal implanted with telomerase-immortalized meningioma cells obtained from a benign tumor.

### Definitions

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

A subject, as defined herein, is an animal such as a vertebrate or invertebrate organism. In other embodiments, the subject is a mammal such as a domesticated farm animal (e.g., cow, horse, pig) or pet (e.g., dog, cat). More preferably, the subject is a human. A subject at risk is a subject who has been determined to have an above-average risk that a subject will develop cancer, which can be determined, for example, through family history or the detection of genes causing a predisposition to developing cancer.

Treat”, “treating”, and “treatment”, etc., as used herein, refer to any action providing a benefit to a subject at risk for or afflicted with a condition or disease such as cancer, including improvement in the condition through lessening or suppression of at least one symptom, delay in progression of the disease, prevention or delay in the onset of the disease, etc. The subject may be at risk due to exposure to carcinogenic agents, being genetically predisposed to disorders characterized by unwanted, rapid cell proliferation, and so on. “Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses.

### Meningioma Animal Models and Tumor Cell Lines

In one aspect, the invention provides a method of generating a tumor cell line. The method includes immortalizing meningioma cells obtained from a benign tumor by introducing telomerase reverse transcriptase to provide telomerase-immortalized meningioma cells and passaging the telomerase-immortalized meningioma cells in growth medium together with untransformed meningioma cells until the untransformed cells undergo senescence to provide a tumor cell line comprising telomerase-immortalized meningioma cells.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue, such as the bone marrow, and causes large numbers of abnormal blood cells to be produced and enter the bloodstream. Lymphoma and multiple myeloma are cancers that begin in the cells of the immune system. Cancer which has metastasized will still retain traits associated with its tissue of origin.

A tumor is a mass of cancer cells. Tumors are generally characterized as either benign tumors or malignant tumors. As is well-known to those skilled in the art, benign tumors usually grow more slowly and do not spread toother parts of the body. Meningiomas are typically benign. Malignant tumors, on the other hand, tend to grow rapidly and can spread to other parts of the body through metastasis.

Meningioma, also known as meningeal tumor, is typically a slow-growing cancer that forms from the meninges, the membranous layers surrounding the brain and spinal cord. Risk factors for meningioma include exposure to ionizing radiation, family history, and neurofibromatosis type 2-related schwannomatosis (NF2 or NF2-SWN). Meningiomas arise from arachnoidal cap cells, most of which are near the vicinity of the venous sinuses, and this is the site of greatest prevalence for meningioma formation. In some embodiments, the meningioma cells are NF2-deficient. The NF2 gene is a tumor suppressor gene that produces a protein called merlin, which regulates cell growth and adhesion. NF2-deficient cells exhibit a decreased level of NF2 protein expression, including a total lack of expression [see Wang et al., Adv Exp Med Biol. 1416, 137-158 (2023)].

Cell lines consist of immortalized cell populations with the ability to divide indefinitely. This is typically due to immortalization in the laboratory or because the cell line is derived from a tumorigenic source from a patient or animal. Cell lines can be maintained in culture for a long time while retaining specific characteristics and functions and are useful, for example, for in vitro studies and drug screening.

The method includes the step of obtaining meningioma cells from a benign tumor. The tumor specimen can be obtained by any suitable means, including biopsy or surgical resection from the brain of a subject having meningioma. Once the meningioma cell tumor specimen has been obtained, it can be prepared for cell culture by, for example, placing the cells in appropriate growth media.

In some embodiments, the meningioma cells are human meningioma cells. In further embodiments, the cells are obtained from a subject having neurofibromatosis type 2-related schwannomatosis (NF2-SWN or NF2 in short). a diagnosis of NF2 is established in a proband with bilateral vestibular schwannomas, an identical NF2 pathogenic variant identified in two or more anatomically distinct NF2-related tumors, or a combination of clinical and molecular criteria that fulfill the consensus diagnostic criteria [see Forde et al., J Med Genet., 61(9): 856-860 (2024)].

The method includes transfecting or transducing meningioma cells obtained from a benign tumor by introducing telomerase reverse transcriptase to provide telomerase-immortalized meningioma cells. Telomerase reverse transcriptase (TERT) is an enzyme that helps maintain telomeres and genome stability, and is expressed by many tumors [about 90%; Zhang et al., Genes & Development. 13 (18): 2388-99 (1999)] since it helps immortalize cells [Kang M & Park N., Methods Mol Biol., 371:151-65 (2007)]. In some embodiments, the TERT is human TERT (hTERT). In some embodiments, the immortalized meningioma cells can also be transfected or transduced to express luciferase.

Introduction of TERT into meningioma cells can be accomplished using transfection or transduction, using a variety of methods well known in the art including for example, electroporation, microparticle bombardment, microinjection, viral transduction, and calcium phosphate treatment. Kits for immortalizing cell lines using TERT are commercially available. See for example GeneCopoeia™ or Creative Biolabs®. A preferred method of transduction is the use of a retroviral or lentiviral vector to introduce the TERT gene into meningioma cells, as described in Example I herein.

In a further aspect, the present invention provides a tumor cell line comprising telomerase-immortalized meningioma cells obtained from a benign tumor. A tumor cell line, which can also be referred to as a cancer cell line, is a cell line based on cancer cells isolated from a tumor. Tumor cell lines can be used for drug screening, studying tumor development, how the immune system responds to tumors, and for studying tumor-targeted therapy. A well-known example of tumor cell lines are the American Type Culture Collection (ATCC) cancer panels, which are a variety of cell lines possessing unique genomic and expression signatures that can be easily grown using well known media formulations.

In some embodiments, the tumor cell line is prepared by immortalizing meningioma cells obtained from a benign tumor by introducing telomerase reverse transcriptase to provide telomerase-immortalized meningioma cells and passaging the telomerase-immortalized meningioma cells in growth medium together with untransformed meningioma cells until the untransformed cells undergo senescence to provide a tumor cell line comprising telomerase-immortalized meningioma cells.

In some embodiments, the meningioma cells of the tumor cell line are NF2-deficient. In further embodiments, the meningioma cells are human meningioma cells. In yet further embodiments, the cells are obtained from a subject having NF2-SWN.

In a further aspect, the present invention provides an animal model of NF2-deficient meningioma. The animal model includes a mammal implanted with telomerase-immortalized meningioma cells obtained from a benign tumor.

An animal model is a non-human species that can be used in biomedical research because it can mimic aspects of a biological process or disease found in humans. Animal models are sufficiently like humans in their anatomy, physiology or response to a pathogen that researchers can extrapolate the results of animal model studies to better understand human physiology and disease. A non-human mammalian animal model including implanted meningioma cells can be referred to as a xenograft model. The present invention makes use of a mammalian animal model. Examples of mammals suitable for use as animal models include rats, mice, dogs, cats, pigs, rabbits, and non-human primates. In some embodiments, the mammal is a mouse.

The animal model is prepared by implanting telomerase-immortalized meningioma cells obtained from a benign tumor into a mammal. The meningioma cells can be implanted at various locations, depending on the type of animal model desired. For example, in heterotopic models, the meningioma cells can be implanted subcutaneously, in the flank, or subrenaly. In other embodiments, an orthotopic animal model is prepared in which the telomerase-immortalized meningioma cells are implanted at the base of the skull of the animal model (e.g., mouse). A detailed description of a method of implanting meningioma cells at the base of the skull of a mouse is provided in Example 1 herein.

In some embodiments, the telomerase-immortalized meningioma cells are human telomerase-immortalized meningioma cells. In further embodiments, the telomerase-immortalized meningioma cells are prepared by transfecting meningioma cells obtained from a benign tumor with telomeres reverse transcriptase to provide telomerase immortalized meningioma cells and passaging the telomerase immortalized meningioma cells in growth medium together with untransformed meningioma cells until the untransformed cells undergo senescence.

### Methods of Treating Meningioma

In another aspect, the invention provides a method of treating meningioma in a subject in need thereof by administering a therapeutically effective amount of a target drug. A target drug is a drug determined to be effective for treating meningioma using the animal model described herein. The effectiveness of cancer treatment may be measured by evaluating a reduction in tumor load. The reduction in tumor load may be represent a direct decrease in mass, or it may be measured in terms of tumor growth delay, which is calculated by subtracting the average time for control tumors to grow over to a certain volume from the time required for treated tumors to grow to the same volume. In some embodiments, the target drug is administered together with a pharmaceutically acceptable carrier. In some embodiments, the meningioma is NF2-deficient meningioma.

In some embodiments, the target drug is brigatinib. Brigatinib, also known as Alacensa® or Alunbring®, is an ALK inhibitor with a known structure and method of synthesis that has been approved for use in treating lung cancer. See Spencer et al., Ann Pharmacother., 53(6): 621-626 (2019).

Candidate agents may be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers [see, for instance, Polin et al., Investig. New Drugs, 15:99-108 (1997)]. Results are typically compared between control animals treated with candidate agents and the control littermates that did not receive treatment. Transgenic animal models are also available and are commonly accepted as models for human disease [see, for instance, Greenberg et al., Proc. Natl. Acad. Sci. USA, 92:3439-3443 (1995)]. Candidate agents can be used in these animal models to determine if a candidate agent decreases one or more of the symptoms associated with the cancer, including, for instance, tumor size, cancer metastasis, cancer cell motility, cancer cell invasiveness, or combinations thereof.

### Formulation and Administration

The present invention includes administering one or more compounds in a pharmaceutical composition. Examples of pharmaceutical compositions include those for oral, intravenous, intramuscular, subcutaneous, inhalation, or intraperitoneal administration, or any other route known to those skilled in the art, and generally involves providing a compound formulated together with a pharmaceutically acceptable carrier. Accordingly, in some embodiments the compound is administered together with a pharmaceutically acceptable carrier.

In some embodiments, the compound is administered orally. When preparing the compounds described herein for oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitoneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

The dosage form and amount can be readily established by reference to known treatment or prophylactic regiments. The amount of therapeutically active compound that is administered and the dosage regimen for treating a disease condition with the compounds and/or compositions of this invention depends on a variety of factors, including the age, weight, sex, and medical condition of the subject, the severity of the disease, the route and frequency of administration, and the particular compound employed, the location of the unwanted proliferating cells (for the cancer and antimicrobial indications), as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

For example, the maximum tolerated dose (MTD) for anticancer compounds can be determined in tumor-free athymic nude mice. Agents are prepared as suspensions (e.g., in vehicle containing 90% polyethylene glycol 300 and 10% 1-methyl-2-pyrrolidinone; 3 animals/group) by oral gavage at doses of 0, 25, 50, 100 and 200 mg/kg once daily for 14 days. Body weights, measured twice weekly, and direct daily observations of general health and behavior will serve as primary indicators of drug tolerance. MTD is defined as the highest dose that causes no more than 10% weight loss over the 14-day treatment period.

The compounds can also be provided as pharmaceutically acceptable salts. The phrase “pharmaceutically acceptable salts” connotes salts commonly used to form alkali metal salts and to form addition salts of free acids or free bases. The nature of the salt is not critical, provided that it is pharmaceutically acceptable. Suitable pharmaceutically acceptable acid addition salts of the compounds may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic, and sulfonic classes of organic acids, examples of which include formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, ambonic, pamoic, methanesulfonic, ethanesulfonic, benzenesulfonic, pantothenic, 2-hydroxyethanesulfonic, toluenesulfonic, sulfanilic, cyclohexylaminosulfonic, stearic, algenic, g-hydroxybutyric, galactaric, and galacturonic acids. Suitable pharmaceutically acceptable base addition salts of the compounds described herein include metallic salts made from aluminum, calcium, lithium, magnesium, potassium, sodium, and zinc. Alternatively, organic salts made from N, N′-dibenzylethylenediamine, chloroprocaine, choline, diethanolamine, ethylenediamine, meglumine (N-methylglucamine) and procaine may be used form base addition salts of the compounds described herein. All of these salts may be prepared by conventional means from the corresponding compounds described herein by reacting, for example, the appropriate acid or base with the compound.

An example has been included to more clearly describe a particular embodiment of the invention and its associated cost and operational advantages. However, there are a wide variety of other embodiments within the scope of the present invention, which should not be limited to the particular example provided herein.

### EXAMPLE

### Example 1: Combining brigatinib with mTOR Inhibition to Effectively Treat NF2-Associated and NF2-Deficient Sporadic Meningiomas

### Introduction

Constituting ~40% of primary intracranial tumors, meningiomas, which originate from meningothelial cells of the arachnoid layer lining the brain and spinal cord, are the most common brain tumors [Ostrom et al., Neuro Oncol., 24(Suppl 5): v1-v95 (2022)]. These tumors can arise at the convexity, the skull base, and along the spine, and may occur at any age, but the incidence increases progressively with age [Wiemels et al., J Neurooncol., 99:307-14 (2010)]. The majority (~80%) of meningiomas are histologically benign (WHO grade 1), whereas the remaining are atypical (grade 2) and anaplastic (grade 3). Benign meningiomas may grow over years without causing symptoms. However, depending on the size and location of growth, they can cause significant morbidity, including seizures, vision loss, neurological deficits, cranial nerve palsy, speech dysfunction, and motor weakness [van Alkemade et al., Neuro Oncol., 14:658-666 (2012)]. Despite high prevalence, meningiomas are under-studied, and presently an FDA-approved medical therapy is not available.

While most meningiomas occur sporadically, these tumors are frequently found in patients with neurofibromatosis type 2 (NF2)-related schwannomatosis (NF2-SWN), a highly-debilitating tumor suppressor syndrome in which affected individuals develop bilateral vestibular schwannomas and multiple meningiomas [Plotkin et al., Genet Med., 24:1967-1977 (2022)]. Currently, surgical resection is the primary treatment strategy for symptomatic or rapidly growing meningiomas. However, surgical removal of meningiomas can damage adjacent normal brain tissue and cause additional neurological deficits. Patients with NF2-SWN frequently develop multiple meningiomas, and surgical excision in these patients is often difficult, especially for tumors located along the skull base. Radiotherapy may be used to manage inoperable or recurrent cases, but radiation treatment increases the risk of malignant transformation or secondary cancers within the radiation field. These drawbacks in the current standard of care underscore the importance of identifying an effective medical therapy that stops tumor growth or completely eliminates meningiomas.

NF2-SWN is caused by biallelic inactivation of the NF2 gene [Rouleau et al., Nature, 363:515-521 (1993)]. Intriguingly, ~50-60% of sporadic meningiomas also harbor NF2 mutations, suggesting an important role of NF2 loss in tumorigenesis. The NF2 gene encodes the tumor suppressor protein merlin (for moesin, ezrin, and radixin-like protein), which is a member of the protein 4.1, ezrin, radixin, moesin (FERM) superfamily, acting as a linker between the plasma membrane and actin cytoskeleton. Merlin has been shown to interact with adherens junction components, mediating contact inhibition of cell proliferation [Lallemand et al., Oncogene, 28:854-865 (2009)]. Merlin can block ligand-mediated internalization of the epidermal growth factor receptor (EGFR)/ErbB family members, other receptor tyrosine kinases (RTKs), such as the insulin-like factor-1 receptor (IGF-1R) and platelet derived growth factor receptor-α (PDGFR-α), and membrane-tethered non-RTKs, like focal adhesion kinase (FAK) [Curto et al., J Cell Biol., 177:893-903 (2007)]. Merlin may also inhibit the delivery of membrane receptors back to the plasma membrane. Further, merlin can restrain the mitogen-activated protein kinase (MAPK), phosphoinositide 3-kinase (PI3K)/AKT/mammalian target of rapamycin (mTOR), FAK-Src, and Hippo signaling pathways [Petrili and Fernandez-Valle, Oncogene, 35:537-548 (2016)]. In addition, merlin can suppress activation of the p21-activated kinase (PAK), and reciprocally, PAK can phosphorylate and inactivate merlin [Shaw et al., Dev Cell., 1:63-72 (2001)]. These results suggest that loss of merlin disrupts multiple signaling pathways, leading to tumorigenesis and that these pathways are potential therapeutic targets for NF2-deficient tumors.

To identify an effective targeted therapy for NF2-deficient meningioma, we previously generated an orthotopic, quantifiable meningioma model [Burns et al., Methods Mol Biol., 1427:59-72 (2016)]. We showed that the Ben-Men-1 cell line established from a sporadic grade-1 meningioma [Püttmann et al., Lab Invest., 85:1163-1171 (2006)] is NF2-null. By generating luciferase-expressing Ben-Men-1-LucB cells and stereotactically injecting them into the skull base of immunodeficient mice, followed by noninvasive bioluminescence imaging (BLI), we showed that Ben-Men-1-LucB cells established intracranial xenografts that grew slowly over time. As a member of the Synodos for NF2 Consortium in collaboration with the National Center for Advancing Translational Sciences (NCATS), we used this model to identify several targeted drugs/drug combinations that potently inhibited tumor growth or caused tumor regression [Chang et al., PLoS One, 16: e0252048 (2021)]. Among the top synergistic drug combinations identified from high throughput screening of an oncology compound library in NF2-defcient vs -expressing meningioma cells, we showed that while the multi-kinase inhibitor dasatinib (BMS-354825) exhibited some anti-tumor activity, the dual mTORC1/2 inhibitor INK128 (sapanisertib, TAK-228, MLN0128) alone and its combination with dasatinib (SPRYCEL®) elicited stronger suppression of meningioma growth [Angus et al., Neuro Oncol., 20:1185-1196 (2018)]. We also demonstrated that as monotherapies, brigatinib (ALUNBRIG®, AP-26113), a multi-kinase inhibitor originally designed as an anaplastic lymphoma kinase (ALK) inhibitor (Siaw et al., Oncotarget., 7:29011-29022 (2016)), caused tumor shrinkage, while the allosteric AKT inhibitor MK2206 only modestly suppressed tumor growth. Combining brigatinib with MK2206 further shrank tumors to smaller sizes following treatment. Importantly, we found that NF2-deficient meningiomas and schwannomas did not express ALK and that brigatinib inhibited multiple RTKs and non-RTKs, including FAK, in these tumor cells. These findings led to a phase II clinical trial of brigatinib in patients with NF2-SWN with progressive vestibular schwannoma, non-vestibular schwannoma, meningioma, and ependymoma (NCT04374305). The trial demonstrated the greatest benefit of brigatinib for meningioma and non-vestibular schwannoma and decreased growth rates for all tumor types [Plotkin et al., N Engl J Med., 390:2284-2294 (2024)].

While Ben-Men-1 represents the first NF2-deficient, grade-1 meningioma cell line immortalized by only using telomerase, it was derived from a sporadic tumor [Püttmann et al., Lab Invest., 85:1163-1171 (2006)]. Curiously, compared to sporadic low-grade meningiomas, tumors in patients with NF2-SWN often exhibit more aggressive growth behavior and are more likely to become symptomatic and recur after resection (Blakeley et al., Am J Med Genet A., 158A: 24-41 (2012)]. Presently, the biology behind these differences is not understood. Also, it is not known whether sporadic and NF2-SWN-related meningiomas respond differently to therapies. The availability of a meningioma cell line established from an NF2-SWN tumor should allow us to compare their tumor biology and enhance NF2 therapeutic development.

In this study, we report the generation of the first telomerase-immortalized NF2-SWN-related meningioma cell line AG-NF2-Men. Since brigatinib and INK128 individually display anti-tumor activities in NF2-deficient tumors [Chang et al., PLoS One, 16: e0252048 (2021)], we explored the possible anti-tumor synergy of combining these two targeted agents. We showed that in both the AG-NF2-Men and Ben-Men-1 models, the combination of brigatinib and INK128 exhibited synergistic growth-inhibitory activity. Combined brigatinib+INK128 treatment more effectively suppressed RTK-mediated phosphorylation of AKT and the mTOR target 4EBP1 (eukaryotic translation initiation factor 4E [eIF4E]-binding protein 1), as well as eliciting major changes in the expression of genes including the upstream regulators of several signaling networks important for meningioma growth. We also generated luciferase-expressing AG-NF2-Men-Luc2 cells and showed that they readily established intracranial tumor xenografts in immunodeficient mice. Using both the intracranial AG-NF2-Men-Luc2 and Ben-Men-1-LucB meningioma models, we demonstrated that the brigatinib+INK128 combination enhanced anti-tumor effects.

### Materials and Methods

Compounds. Brigatinib (CAS: 1197953-54-0) and INK128 (CAS: 1224844-38-5) were synthesized by Proactive Molecular Research (Alachua, Florida). Based on liquid chromatography-mass spectrometry and nuclear magnetic resonance analysis, their purities were >98%. For in vitro studies, these compounds were dissolved in DMSO as 10 mM stocks, and aliquots stored at −20° C. For mouse dosing, brigatinib was formulated as 5 mg/mL solution in 90% polyethylene glycol 300 and 10% 1-methyl-2-pyrrolidinone, and INK128 as 0.075 mg/mL solution in 5% 1-methyl-2-pyrrolidinone, 15% polyvinylpyrrolidone K30, and 80% water. For combination treatment, brigatinib and INK128 were prepared as 2× concentrates in each respective vehicle, and equal amounts of each drug solution were mixed to obtain the final concentration of 5 mg/mL of brigatinib and 0.075 mg/mL of INK 128. All mice received 0.1 mL of each formulated drug or drug combination per 10 g of mouse weight once a day by oral gavage.

Tumor procurement, FoundationOne® test, and immunohistochemistry (IHC). A meningioma specimen was procured from a 30-year-old male patient with NF2-SWN with informed consent according to The Ohio State University Institutional Review Board-approved Human Subjects protocol. The tumor was surgically resected from the left cerebellopontine angle region and appeared as a firm, light tan-to-brown, fibrous mass of 2.0×1.2×0.8cm in size. A piece of tumor was fixed in formalin and processed for paraffin sectioning and hematoxylin-eosin staining. A certified neuropathologist diagnosed the tumor as a WHO grade-1 meningioma showing proliferation of meningothelial cells with prominent psammoma body formation. To detect genomic alterations, tumor sections were submitted to Foundation Medicine for a FoundationOne® Heme next-generation sequencing test, which interrogates 406 genes commonly mutated in human cancers as well as selected introns of 31 genes involved in rearrangements, in addition to RNA sequencing (RNA-seq) of 265 genes. Also, tumor sections also immunostained with antibodies against phospho-ErbB3[Y¹²⁸⁹] (p-ErbB3[Y¹²⁸⁹]; #4791), p-Erk1/2[T²⁰²/Y²⁰⁴] (#4370), p-AKT[S⁴⁷³] (#4060; all from Cell Signaling Technology), p-FAK[Y³⁹⁷] (#700255, ThermoFisher), epithelial membrane antigen (EMA; #HY500076, Applied Biological Materials), CD163 (#CM353; Biocare Medical), and c-MYC (#RA0226, ScyTek Laboratories).

Immortalization, telomerase assay, and fluorescence in situ hybridization (FISH). A piece of freshly-resected NF2-SWN meningioma tissue was placed in Dulbecco's Modified Eagle's medium (DMEM; ThermoFisher) and transported to lab. Primary meningioma cell culture was prepared and grown in DMEM plus 10% heat-inactivated fetal bovine serum (FBS; R&D Systems) as previously described (Burns and Chang, Methods Mol Biol., 1427:59-72 (2016)). For immortalization, human telomerase reverse transcriptase (hTERT)-expressing retroviruses were produced by co-transfecting pLNCX2-hTERT and pVSV-G into GP 2-293 packaging cells (Takara Bio). After 24 and 48 h, the supernatant containing retroviral particles was collected and filtered through a 0.45-μm filter (Corning). Upon reaching confluence, primary meningioma cells were split into two dishes. The next day, one dish was infected with 1 mL of supernatant containing hTERT-expressing retroviruses in the presence of 8 mg/mL polybrene, and the other dish incubated only with polybrene (MilliporeSigma). Following overnight incubation at 37° C., the culture medium was replaced, and both dishes of cells were grown and passaged in medium without G418 selection until immortalized cells emerged and non-transduced cells senesced. The hTERT-immortalized cells, designated AG-NF2-Men, were grown in G418-containing medium and subcloned.

The telomerase activity in primary and immortalized AG-NF2-Men cells was measured using the Telomerase Activity Quantification qPCR Assay Kit (ScienCell). Real-time quantitative PCR amplification of telomerase repeats was performed according to manufacturer's instructions using a Bio-Rad CFX96 Touch thermocycler. The telomerase activity of immortalized AG-NF2-Men cells was estimated relative to primary meningioma cells by the ΔCt method. To confirm amplification of a specific product, melt curve analysis was performed from 65 to 95° C. in 0.5° C. increments, and the ~75-bp PCR product electrophoresed on a 3.5% agarose gel in Tris-acetate/EDTA buffer.

FISH analysis was performed using the Vysis EWSR1 (22q12) Dual Color Break Apart Rearrangement FISH Probe Kit (Abbott Molecular) on nuclei from the primary and immortalized meningioma cells according to the manufacturer's instructions. The probe kit contains two DNA probes from the chromosome 22q12 region in which the NF2 gene is located. The first is a 497-kb probe labeled in Spectrum Orange and flanking the 5′ side of the EWSR1 gene. The second is a 1,100-kb DNA labeled in Spectrum Green and extends from the 3′ end of the EWSR1 gene to the sequence beyond the NF2 gene. In a cell with two intact copies of chromosome 22, a two-red: green fusion signal pattern is observed, while only one red: green fusion signal is seen in the cell with only one chromosome 22. Dual color probe-hybridized cells were analyzed by a board-certified cytogeneticist.

Resazurin assays, drug combination matrix arrays, and cell counting. AG-NF2-Men cells were seeded in 96-well plates (Sarstedt) at 4,000 cells/well and allowed to adhere overnight. Brigatinib and INK128 were added to cells in culture medium as 9-point, 2-fold serial dilutions with 4-12 replicate wells per treatment dose. Cell proliferation was measured after 3 days by resazurin assay (Burns et al., 2013). GraphPad Prism v10 was used for plotting the dose-response curves using nonlinear regression analysis for curve fitting, and the mean absolute IC50 (50% inhibitory concentration) values estimated from the curves. For drug combinations, AG-NF2-Men (4,000 cells/well) and Ben-Men-1 cells (2,000 cells/well) were seeded as 8×8 matrices in 96-well plates. The following day, brigatinib and INK128 were arrayed in combination using a 7-point, 2-fold dilution series. Cell proliferation of Ben-Men-1 cells was measured by resazurin assay after 3 days of treatment. Due to slower population doubling times, AG-NF2-Men cell proliferation was assessed after being treated for 7 days or longer. SynergyFinder v3.0 was used to calculate synergy scores according to the Loewe additivity model. The growth-inhibitory synergy of the brigatinib+INK128 combination in AG-NF2-Men cells was also assessed by cell counting. Cells were seeded at 2,000 cells/cm² in 6-well plates (Sarstedt) and treated with 1X IC₅₀ of brigatinib, INK128, or brigatinib+INK 128, or DMSO as controls. Duplicate wells of treated cells were counted by hemocytometer every 3-4 days, and media and compounds were refreshed on unharvested wells.

Western blots and immunofluorescence staining. Actively growing AG-NF2-Men cells, Ben-Men-1 cells, primary normal meningeal cells (ScienCell), primary meningioma cells from four NF2-SWN tumors, and HeLa cells were lysed in cold Triton X-100 lysis buffer. Equal amounts of cleared protein lysates were resolved on an SDS-polyacrylamide gel, electroblotted onto an Immobilon-FL membrane (MilliporeSigma), and then probed with various indicated antibodies. Primary antibody-bound proteins were detected using IRDye-conjugated secondary antibodies, followed by scanning on an Odyssey CLx Imaging System (LI-COR) using the appropriate fluorescent channel at a resolution of 84 μm. Also, AG-NF2-Men cells were starved overnight in serum-free DMEM. The next day, starved cells were stimulated for 10 minutes with 50 ng/mL of EGF, heregulin (Hrg), or IGF-1 and then lysed for Western blotting as described above. To investigate the effects of drug treatments, subconfluent AG-NF2-Men cells were treated for 1 and 3 days with 1X IC₅₀ of brigatinib, INK128, or the brigatinib/INK128 combination and then lysed as described above. Also, serum-starved cells were pretreated for 2 hours with 1X IC50 of brigatinib, INK128, or brigatinib/INK 128 and then stimulated for 5 minutes with 50 ng/mL of EGF, heregulin (Hrg), or IGF-1, followed by cell lysis and Western blotting. The primary antibodies used include anti-merlin (#12888), EGFR (#4267), p-EGFR[Y1068] (#3777), p-ErbB3[Y¹²⁸⁹] (#4791), IGF-1R (#9750), p-IGF-1R[Y1135/1136] (#3024), AKT (#2920 and #4691), p-AKT[S⁴⁷³] (#4060), p-AKT[T³⁰⁸] (#2965), ERK1/2 (#4695), p-ERK1/2[T²⁰²/Y²⁰⁴] (#4370), 4EBP1 (#9644), p-4EBP1[T⁶⁵] (#9451), p-4EBP1[S^{37/46}] (#2855), GAPDH (#5174) (all from Cell Signaling Technology), desmoplakin 1/2 (#GTX41413, GeneTex), α-tubulin (#sc-322930) and ErbB3 (#sc-285) (both from Santa Cruz Biotechnology).

For immunofluorescence staining, AG-NF2-Men cells were plated at 2×10⁴ cells/cm² on sterile glass coverslips. The following day, cells were washed in phosphate-buffered saline (PBS) and fixed at room temperature (RT) for 30 min in 4% paraformaldehyde. Fixed cells were blocked with 10% bovine serum albumin in PBS, permeabilized for 15 min at RT in 0.1% Triton X-100, and incubated with an anti-vimentin antibody (#ab16700, Abcam) for 2 h at RT and then an Alexa Fluor 488-conjugated goat anti-rabbit secondary antibody (#A11029, Molecular Probes) for 1 h at RT. Following washing with PBS four times, nuclei of stained cells were counterstained for 10 min at RT with 0.2 μg/mL of 4′,6-diamidino-2-phenylindole (DAPI) in PBS. Immunofluorescent images were captured using a Leica DM IRB inverted UV microscope attached to a SPOT digital camera imaging system.

RNA-seq analysis. AG-NF2-Men cells plated at 15,000 cells/cm² in 10-cm dishes were treated in triplicate with 1X IC₅₀ of brigatinib, INK128, or their combination, or an equivalent amount of DMSO (0.01%) in fresh DMEM plus 10% FBS. After 24-h incubation, cells were scraped off dishes, washed twice with cold PBS, and flash-frozen at −80° C. until shipment on dry ice to MedGenome (Foster City, CA) for RNA-seq analysis. Total RNA isolation was performed using the Maxwell RSC simplyRNA Cells kit (Promega). Libraries were prepared with the Illumina TruSeq stranded mRNA sample preparation kit, and 100-bp paired-end reads were sequenced on an Illumina NovaSeq6000 sequencing system. After excluding non-poly(A)-tailed RNA sequences with Bowtie2 (v.2.5.1), reads were mapped by STAR v2.7.3a to the GRCh37/hg19 human reference genome. Raw reads estimated by HTSeq v0.11.2 were normalized with DESeq2. Gene expression levels in FPKM (fragments transcript kilobase per million) were determined in Cufflinks v2.2.1. Differentially expressed genes (DEGs) were calculated by DESeq2. Significant DEGs were defined as transcripts with absolute log2 fold-changes (log2 FC)≥1 and adjusted P-values (Padj)≤0.01. Overlapping DEG were graphed as proportional Venn diagrams using DeepVenn (https://deepvenn.com). Volcano plots for DEGs were constructed using the Galaxy Project bioinformatics website. Predicted upstream signaling regulators activated or inhibited in drug-treated AG-NF2-Men cells were ascertained by the Ingenuity Pathway Analysis (IPA) Upstream Analysis module (Qiagen), following Core Analysis of the transcriptomic dataset with the cutoffs: Padj<0.01, absolute log2 FC>1, mean normalized read counts>5.

Generation of luciferase expressing AG-NF2-Men cells and skull-base meningioma xenograft models. To generate luciferase-expressing derivatives, immortalized AG-NF2-Men cells were infected with Lenti-CMV-Luc lentiviruses (Qiagen). Puromycin-resistant colonies were selected and assessed for luciferase activity using the One-GLO™ Luciferase Assay System (Promega). The clone AG-NF2-Men-Luc2, which expressed the highest luciferase activity, was tested for their ability to establish xenografts by injecting cells (5×10⁵) subcutaneously in the flank or orthotopically in the skull base of 8-to-12 week-old NSG mice (NOD-SCID gamma or NOD. Cg-Prkdcscid Il2rgtmlWjl/SzJ; The Jackson Laboratory). All animal procedures were performed according to the protocol approved by the Institutional Animal Care and Use Committee of Nationwide Children's Hospital. Injected mice were monitored by weekly bioluminescence imaging (BLI) using an IVIS Spectrum In Vivo Imaging System (Revvity). Mice with established intracranial tumors, defined as increased in BL signals over at least two consecutive timepoints, were randomized into different treatment groups and received vehicle, 50 mg/kg of brigatinib, 0.75 mg/kg of INK 128, or their combination (n=5/group) every day by oral gavage. The effects of treatment were monitored by BLI. After 8-week treatment, we stopped treating a cage of AG-NF2-Men-Luc2 xenograft-bearing mice that had received INK128 or the brigatinib+INK128 combination and monitored for possible tumor regrowth for six more weeks. Then, we retreated these mice to determine whether regrown tumors were still drug sensitive.

### Results

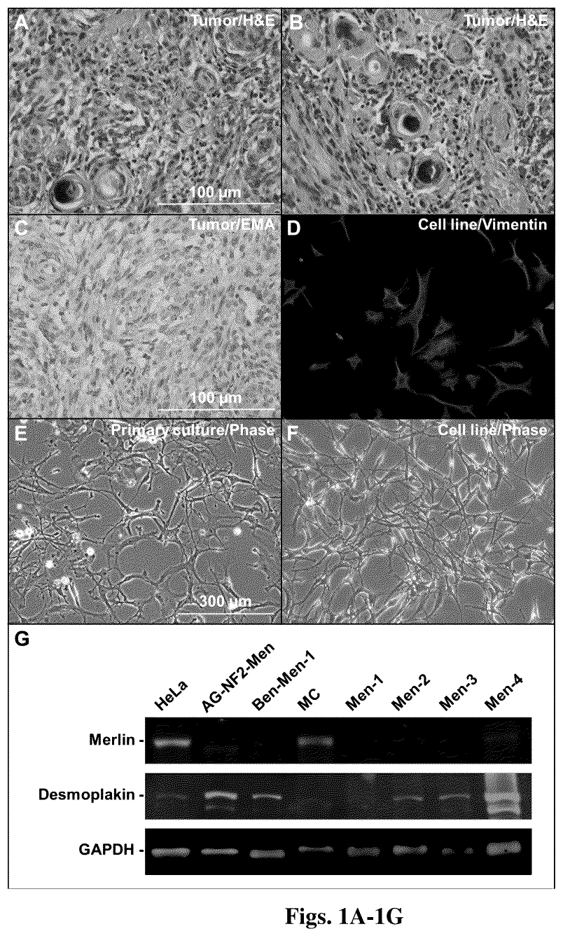

Establishment of the bona fide NF2-SWN meningioma cell line AG-NF2-Men. To generate an NF2-SWN meningioma cell line, we procured a WHO grade-1 meningioma from a patient with NF2-SWN. Histologically, the tumor contained meningothelial cells appearing relatively large and elongated with a whorling architecture and psammoma bodies (FIG. 1A, B) and expressed the meningioma marker EMA (FIG. 1C). There were no atypia or atypical mitosis, and no evidence of malignancy. Despite the patient having previously received several drug treatments in clinical trials, the FoundationOne® Heme test revealed that the tumor exhibited low mutational burden and stable microsatellite status with only two pathogenic variants identified: a splice site mutation (447+1G>C) in the NF2 gene and a duplication of MYC exons 2-3. Due to NF2 loss, the tumor exhibited elevated phosphorylation of merlin-regulated kinases, such as the receptor tyrosine kinase (RTK) ErbB3 and the integrin-linked non-RTK focal adhesion kinase (FAK), as well as their downstream signaling molecules AKT and ERK1/2, compared to normal human sciatic nerve and mouse brain and meninge tissues. Additionally, the tumor exhibited strong cMYC staining in in the nucleus. Also, we observed infiltration of macrophages that are immunopositive for CD163, a marker of M2 macrophages that often comprise pro-tumorigenic microenvironments.

To establish an immortalized cell line, we prepared primary cell culture from this NF2-SWN meningioma and transduced it with hTERT-expressing retroviruses. To enhance successful growth of immortalized cells, transduced cells were allowed to grow and split in complete growth medium without G418 selection until immortalized cells emerged. We reasoned that since primary NF2-deficient tumor cells have poor transduction efficiency and do not grow well in low density, G418 selection immediately following retrovirus infection would eliminate un-transduced cells and leave only few hTERT-transduced cells that might not grow in sparse conditions. Primary meningioma cells usually grow in culture only for 10-15 passages before they undergo senescence and die. However, under no G418 selection, we observed that after 15 passages, some cells in the hTERT-transduced dish continued to grow and formed small colonies surrounded by residual enlarged, flattened, and vacuolating senesced cells. More colonies or islands of cells were seen in subsequent passages, suggesting possible immortalization. Therefore, we added G418 (500 μg/mL) to the culture at passage 18 and found that these colonies were G418-resistant and were able to grow to confluence. These immortalized cells, designated AG-NF2-Men, were subcloned in G418-containing medium and have been grown for more than 100 passages.

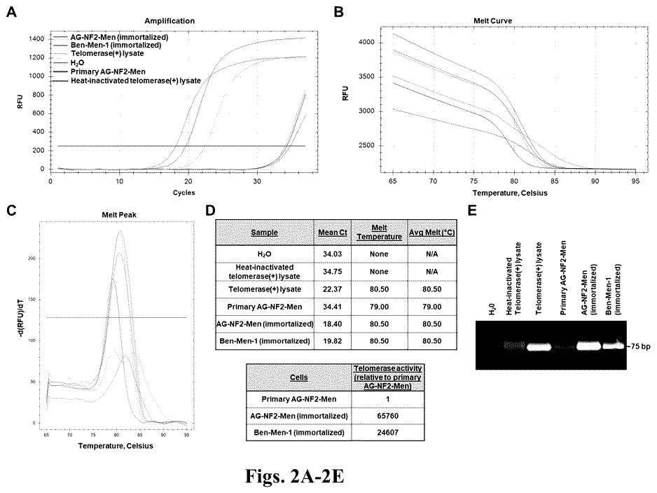

We confirmed telomerase expression in immortalized AG-NF2-Men cells by the telomeric repeat amplification protocol, which detects telomeric repeats added to the 3′ end of a telomerase-specific substrate. Primary non-transduced AG-NF2-Men tumor cells at passage 2 had little or no telomerase activity (the cycle threshold value Ct=34.41), similar to the negative controls with H2O (Ct=34.03) or heat-inactivated telomerase(+) cell lysate (Ct=34.75) (FIGS. 2A-D). Immortalized AG-NF2-Men cells at passage 37 had robust telomerase activity, comparable to that in the Ben-Men-1 cell line (Ct=18.40 vs 19.82, respectively). The telomerase activity in immortalized AG-NF2-Men cells was ~6.6×10⁴-fold higher than that in the primary culture (FIG. 2D). Melt-curve analysis of the telomerase reaction in hTERT-expressing AG-NF2-Men cells showed a single peak with a melting temperature nearly identical to the positive control, indicating specific amplification of the telomerase reaction product (FIG. 2B, C). Gel electrophoresis confirmed the presence of the telomerase amplicon (FIG. 2E). These results indicate that immortalized AG-NF2-Men cells stably re-express high levels of telomerase activity.

The AG-NF2-Men cell line has lost a copy of chromosome 22, is merlin-null, and exhibits characteristic of benign meningioma.

To verify that immortalized AG-NF2-Men cells were derived from the tumor cells in the original NF2-SWN-related meningioma, we first performed FISH analysis to determine the status of chromosome 22 in the primary culture and immortalized cells. Using dual color DNA probes with the red probe covering the 5′ side of the EWSR1 gene and the green probe with sequence extending the 3′ EWSR1 region to that beyond the NF2 gene on chromosome 22q12, we found that the majority of cells in the primary meningioma culture exhibited only one red-green fusion signal, indicating the presence of only one copy of chromosome 22. The primary culture also showed sporadic instances of cells with two copies of chromosome 22, which are likely contaminating normal fibroblasts or other stromal cells present in primary tumor cell cultures. Importantly, immortalized AG-NF2-Men cells displayed uniform chromosome 22 monosomy. Together with the mutational analysis from FoundationOne® test, these results demonstrate that AG-NF2-Men tumor cells have NF2 mutations on one chromosome 22 and a complete loss of the other chromosome 22 likely due to the loss of heterozygosity during tumorigenesis.

The AG-NF2-Men cell line retained several characteristics of the original tumor. It exhibited relatively slow growth, with an estimated population doubling time of ~2.6 days. This growth rate was similar to that of the Ben-Men-1 cell line but markedly slower than that of the NF2-deficient, malignant meningioma cell line KT21-MG1, which has a one-day doubling time. Morphologically, immortalized AG-NF2-Men cells appeared multi-polar with numerous processes resembling lamellipodia and filopodia characteristic of NF2-SWN related tumor cells. James et al., Mol Cell Biol., 29:4250-4261 (2009). Similar to the primary AG-NF2-Men culture, they tended to grow in a spider-web pattern reminiscent of leptomeningeal cells (FIG. 1E, F), indicating that immortalization by telomerase did not significantly change cell morphology. RNA-seq detected RNA expression for the desmoplakin (DSP), other desmosomal proteins desmoglein 2 (DSG2), desmocollin 3 (DSC3), and plakophilins 2 and 4 (PKP2 and PKP4), as well as epithelial membrane antigen (EMA) and vimentin (VIM) immortalized AG-NF2-Men cells. Consistently, Western blots detected the expression of vimentin and desmoplakin 1/2 proteins (FIG. 1D, G), which are characteristically co-expressed by arachnoidal and meningioma cells. Baia et al., J Neurooncol., 78:113-121 (2006). However, AG-NF2-Men did not express DSG3 RNA and protein as has been reported in other meningiomas. Akat et al., Acta Neuropathol., 106; 337-347 (2003). Also, in line with FoundationOne® and FISH tests, the AG-NF2-Men cell line did not have any merlin protein (FIG. 1G) but expressed several merlin-regulated growth-factor receptors EGFR, ErbB3, and IGF-1R. All three RTKs underwent robust ligand-mediated auto-phosphorylation followed by downstream activation of p-AKT and p-ERK1/2. RNA-seq analysis of RTK and ligand pairs showed that immortalized AG-NF2-Men cells expressed a low level of NRG1, which codes for the ErbB3 ligand heregulin, but not EGF and IGF-1. Together, these results indicated that we have established a NF2-SWN related meningioma cell line that retains characteristics of benign meningioma and express several RTKs implicated in driving meningioma growth.

The dual mTORC1/2 inhibitor INK128 synergizes with the multi-kinase inhibitor Brigatinib to block AG-NF2-Men cell proliferation by completely abrogating AKT and 4EBP1 phosphorylation.

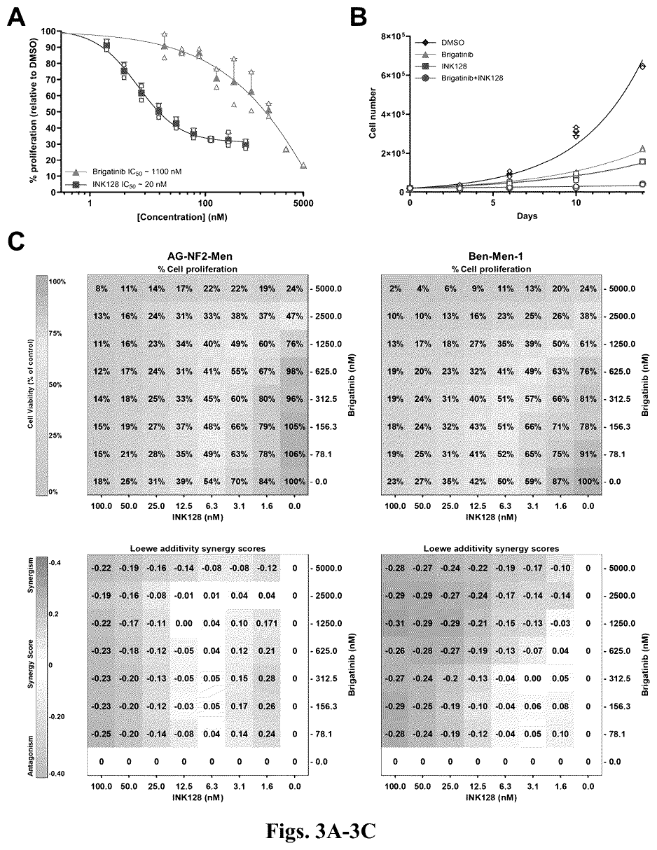

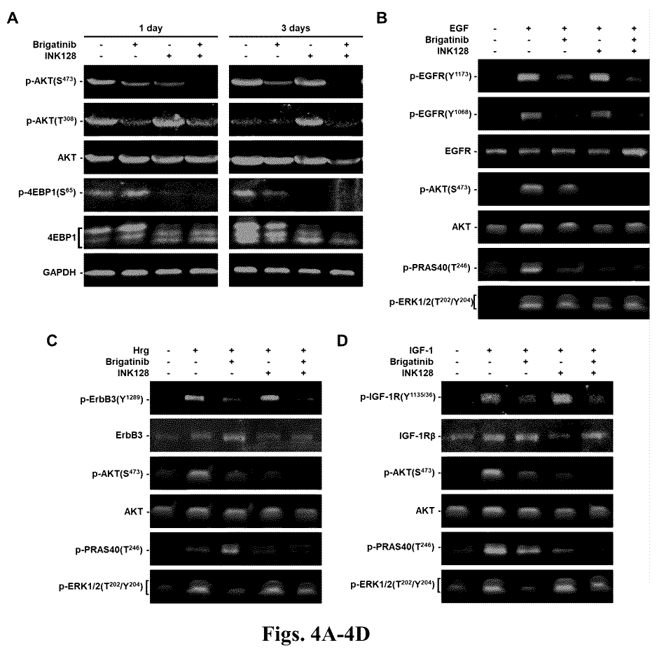

Since loss of NF2 function is associated with deregulated mTORC1/2 signaling (James et al., Mol Cancer Res., 10:649-659 (2012)), we previously showed that the mTOR kinase inhibitor INK128 effectively suppresses the growth of Ben-Men-1 cells in vitro and tumors in the orthotopic, luciferase-expressing Ben-Men-1-LucB model (Angus et al., Neuro Oncol., 20:1185-1196 (2018)). We also found that the multi-kinase inhibitor brigatinib causes tumor shrinkage of intracranial Ben-Men-1-LucB xenografts; however, as a monotherapy, brigatinib did not eliminate Ben-Men-1-LucB cells as treated tumors resumed growth upon cessation of treatment. Therefore, we examined whether combining INK128 with brigatinib would enhance anti-tumor effects in both the AG-NF2-Men and Ben-Men-1 models. Brigatinib and INK128 elicited growth inhibition in AG-NF2-Men cells, with the IC₅₀ values of about 1.1μM and 20 nM, respectively (FIG. 3A). These values were similar to those observed in Ben-Men-1 cells. Bhattacharyya et al., Neurooncol Adv., 2024; 6: vdae 024. To examine the enhanced growth-inhibitory activity of the brigatinib+INK128 combination, we first performed cell counting assays on AG-NF2-Men cells treated for up to 14 days with 1x IC₅₀ of brigatinib, INK128 or their combination. Brigatinib or INK128 alone substantially reduced the growth of AG-NF2-Men cells, and the combined brigatinib+INK128 treatment almost completely arrested their growth (FIG. 3B). We then treated both AG-NF2-Men and Ben-Men-1 cells with brigatinib and INK128 in 8×8 drug matrix arrays for 7 and 3 days, respectively, and measured the effects of drug treatment using resazurin assays. The brigatinib+INK128 combination synergistically inhibited proliferation of both meningioma cell lines with the maximum Loewe synergistic score of −0.25 and −0.31 for AG-NF2-Men and Ben-Men-1 cells, respectively (FIG. 3C).

To understand how brigatinib and INK128 elicited synergistic growth inhibition in meningioma cells, we examined AKT phosphorylation on S473 and T308 in actively growing AG-NF2-Men cells treated with 1x IC₅₀ of brigatinib, INK128, or their combination. Brigatinib treatment partially reduced AKT phosphorylation on both S473 and T308 after one-day treatment, and this partial reduction in p-AKT(S473) and p-AKT(T308) was still observed after three days (FIG. 4A). In contrast, INK128 treatment of AG-NF2-Men cells for one day reduced AKT phosphorylation only on S473, but not on T308, and this effect was transient as the p-AKT(S473) level almost returned to the baseline level by three days. Additionally, INK128 alone slightly increased p-AKT(T308) after one-day treatment, and the increase in p-AKT(T308) was even more pronounced relative to the DMSO-treated control after three days, suggesting the activation of feedback signaling leading to re-phosphorylation of AKT at this residue. Since 4EBP1 is a major target of the AKT/mTORC1 pathway and function as a protein translational repressor that is inactivated by phosphorylation by these kinases, we found that INK128 reduced 4EBP1 phosphorylation at the S65 site, a key phosphorylation site for regulating its binding affinity to eIF 4E. Karim et al., J Biol Chem., 276:20750-20757 (2001). Consistently, an increase in faster-migrating, hypophosphorylated species of 4EBP1 was observed after INK128 treatment. However, brigatinib did not affect the p-EBP1(S65) level after one-day treatment and only moderately decreased p-4EBP(S65) after three days. Thus, when the blots were probed for total 4EBP1, abundant slow-migrating, hyperphosphorylated 4EBP1 species were detected in brigatinib-treated AG-NF2-Men cells after three days, similar to DMSO-treated controls. Importantly, the brigatinib+INK128 combination not only more effectively blocked p-AKT(S473) at both timepoints than either drug alone, but also completely prevented INK128-mediated re-phosphorylation of AKT on T 308 by 3 days. Also, the combination treatment diminished p-4EBP1(S65) phosphorylation after one-day treatment. While brigatinib alone only slightly reduced p-4EBP1, combined brigatinib+INK128 treatment effectively converted the majority of 4EBP1 to a fast-migrating, hypophosphorylated species.

Previously, we showed that brigatinib inhibits multiple merlin-regulated RTKs, including EGFR, ErbB3, and IGF-1R, in Ben-Men-1 cells. Chang et al., PLoS One, 16: e0252048 (2021). Therefore, we investigated the ability of the brigatinib+INK128 combination to suppress ligand-mediated growth signaling from these RTKs. AG-NF2-Men cells, when serum starved for 24 hours, did not express phosphorylated EGFR at its autophosphorylation sites Y1173 and Y1068 and exhibited little or no p-AKT(S473) and its downstream p-PRAS40, as well as p-ERK1/2. While brigatinib treatment did not affect phosphorylation of these signaling molecules in growth-arrested AG-NF2-Men cells, INK128 alone and its combination with brigatinib appeared to slightly enhance ERK1/2 phosphorylation, consistent with previous findings indicating compensatory phosphorylation and activation of ERK1/2 signaling by AKT/mTOR inhibition.

As expected, addition of EGF rapidly induced p-EGFR(Y1173 and Y1068), p-AKT(S473), p-PRAS40, and p-ERK1/2 (FIG. 4B). Brigatinib treatment completely block ligand-mediated phosphorylation of EGFR at Y1068. Intriguingly, brigatinib substantially reduced, but did not eliminate p-EGFR(Y1173), and as a result, some p-AKT and p-ERK1/2 were still detected in treated cells. INK128 treatment did not affect EGFR phosphorylation but completely blocked pAKT and its downstream p-PRAS40. The brigatinib+INK128 combination effectively suppressed p-EGFR, p-AKT, and p-PRAS40j. Like brigatinib alone, the combination-treated cells still exhibited a small but detectable level of p-EGFR(Y1173) and p-ERK1/2 in EGF-stimulated cells. Similar results were observed for ErbB3, IGF-1R, AKT, and PRAS40 phosphorylation in Hrg-and IGF-1-stimulated AG-NF2-Men cells (FIG. 4C, D). In particular, the brigatinib+INK128 combination had greater efficacy at diminishing p-AKT and p-PRAS40 compared to brigatinib alone, and also more effectively reduced ligand-mediated receptor phosphorylation compared to the INK128 treatment.

Collectively, these results suggest that INK128 enhances the ability of brigatinib to suppress RTK-mediated phosphorylation at S473, prevents feedback re-phosphorylation of p-AKT(T308) by INK128, and blocks 4EBP1 phosphorylation, leading to synergistic growth inhibition.

Combining INK128 with brigatinib elicits an exaggerated transcriptomic response with suppression of multiple drivers of meningioma growth.

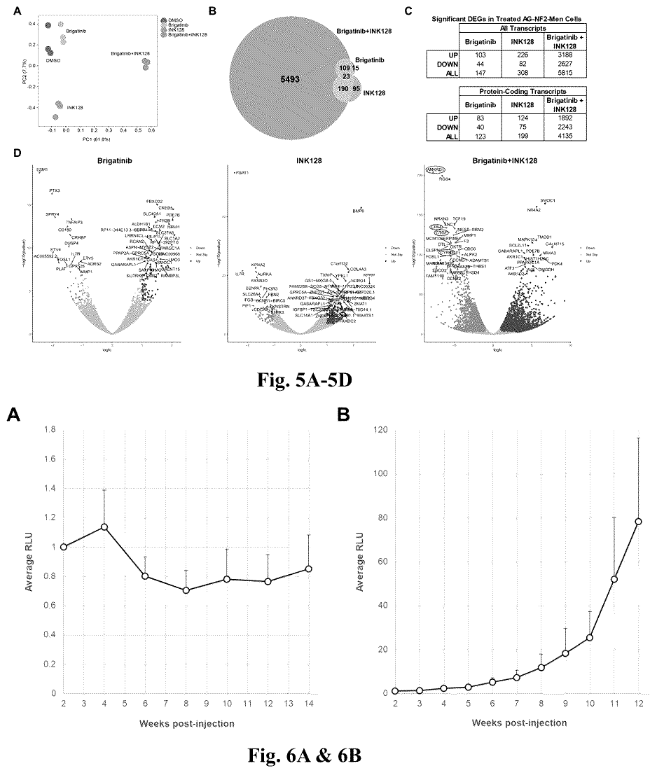

To further investigate the mechanism underlying the enhanced antiproliferative effects of the brigatinib+INK128 combination, we performed RNA sequencing analysis on AG-NF-Men cells treated for one day with 1x IC₅₀ doses of brigatinib, INK128, or their combination. single-agent brigatinib and INK128 only modestly affected gene expression with only 147 and 308 DEGs, respectively (FIG. 5). However, the brigatinib+INK128 combination treatment significantly affected the expression of 5815 genes, with 5493 DEGs exclusive to the combination treatment (FIG. 5B, C). Interestingly, we noticed that four of the top 50 DEGs were the YAP target genes, including ANKRD1, CPA4, CTGF, and CYR61, and their expression was strongly decreased in combination-treated cells (FIG. 5D). Also, the expression of several other YAP-regulated genes, such as AMOTL2, AXL, ANXA3, AJUBA, ANXA1, and CITED2, were found to be reduced, while single-agent brigatinib and INK128 did not significantly affect the expression of these YAP target genes (Padj ≤0.01). In addition, among the top 50 DEGs, the brigatinib+INK128 combination significantly decreased transcripts encoding proteins important for DNA synthesis and cell cycle progression, such as MCM10, CCNE2, and CCND1 (FIG. 5D).

IPA showed that only the combination-treated AG-NF2-Men cells were predicted to have statistically significant inhibition of the EGFR, ErbB3, ErbB4, and NRG1 signaling. These findings are consistent with our observed inhibition of ligand-induced signaling from ErbB3 and EGFR in AG-NF2-Men cells treated with brigatinib alone and its combination with INK128 (FIG. 4). Additionally, activation of AKT, which is an important mitogenic downstream target of the ErbB family and mTOR, was also predicted to be suppressed. Consistent with the reduced transcript levels of multiple YAP transcriptional targets (FIG. 5D), the transcriptional activities of YAP, TAZ, and TEAD were predicted to be reduced. Concomitantly, LATS1/2, which repress YAP, were expected to be activated.

Collectively, our transcriptomic analysis corroborates the results from Western blotting, demonstrating that the brigatinib+INK128 combination more durably suppresses the downstream signaling of their kinase targets as well as the activity of a broad range of mitogenic signaling molecules, leading to growth-inhibitory synergy in treated AG-NF2-Men cells.

The brigatinib+INK128 combination elicits enhanced tumor suppression in Mice Bearing Intracranial Ag-nf2-men-luc2 Xenografts.

To establish a quantifiable animal model for NF2-SWN associated meningioma, we generated luciferase expressing AG-NF2-Men-Luc2 cells by transducing immortalized AG-NF2-Men-1 cells with the Lenti-CMV-Luc lentiviral vector carrying a luciferase-expressing cassette and a puromycin-resistant marker. A puromycin-resistant clone expressing the highest level of luciferase activity (designated AG-NF2-Men-Luc2) was selected for animal implantation. First, we injected AG-NF2-Men-Luc2 cells subcutaneously in the back of NSG mice and monitored tumor growth by weekly BLI for 14 weeks. Intriguingly, these AG-NF2-Men-Luc2 cells did not grow when implanted at the subcutaneous location. FIG. 6A. Then, we stereotactically injected AG-NF2-Men-Luc2 into the skull base of NSG mice. BLI showed that in contrast to those inoculated subcutaneously, these orthotopically implanted xenografts steadily grew over time. FIG. 6B. These results indicated the establishment of a quantifiable, intracranial model for NF2-SWN associated meningioma and suggest that the tumor microenvironment plays an important role in supporting AG-NF2-Men-Luc2 meningioma growth.

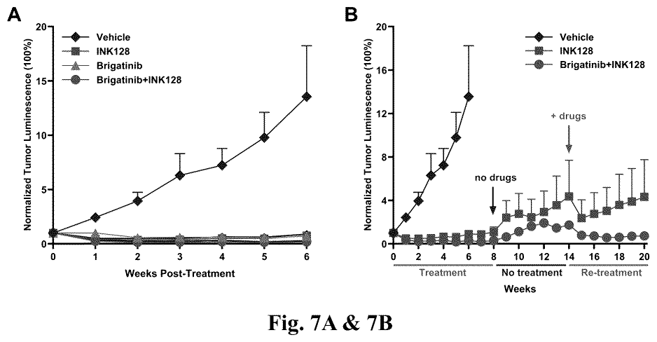

To examine the anti-tumor activities of brigatinib, INK128, and their combination, we generated and treated a cohort of NSG mice bearing established intracranial AG-NF2-Men-Luc2 xenografts. BLI showed that vehicle-treated tumors grew steadily over time with a 13-fold increase of mean tumor-emitted bioluminescence after six weeks (FIG. 7A). In contrast, brigatinib alone caused tumor shrinkage over the six-week treatment period with the average tumor-emitted bioluminescence signal reduced to about 71% of pretreated tumors. INK128 alone also effectively blocked meningioma growth particularly in the first two weeks, and then treated tumors showed slight growth. Nevertheless, treatment with INK128 for six weeks resulted in reduction of tumor-emitted bioluminescence to ~75% of that of pretreated tumors. Notably, combined treatment with brigatinib and INK128 elicited the strongest effect on tumor regression with treated tumor reduced to ~18% of pretreated tumors over six weeks.

To assess potential tumor regrowth after cessation of treatment and the efficacy of re-treatment, we stopped treating one cage of mice in the groups that had been treated with INK128 alone or the brigatinib+INK128 combination for eight weeks and continued monitoring tumor growth by BLI for another six weeks. Upon cessation of treatment, both INK128- and combination-treated tumors exhibited some regrowth; however, the combination-treated tumors regrew at a much slower rate than those treated with INK128 alone (FIG. 7B). To examine the effects of re-treatment, we restarted treatment on these mice for six more weeks. Encouragingly, tumor shrinkage was observed when the treatment was re-initiated. In particular, the regrown tumors shrank substantially upon re-treatment with the brigatinib+INK128 combination, while the tumors re-treated with INK128alone showed only transient tumor shrinkage on the first week. Collectively, these results suggest that the brigatinib and INK128 combination can be repeatedly used to treat NF2-SWN associated meningiomas.

### Discussion

To help identify novel medical therapies beneficial for patients with NF2-SWN related meningiomas, it is important to use an animal model that recapitulates benign growth characteristics of these intracranial tumors. While mice with conditional Nf2 inactivation in leptomeningeal cells or in prostaglandin D2 synthase (PGDS)-expressing meningeal progenitor cells develop meningiomas [Kalamarides et al., Oncogene, 30:2333-2344 (2011)], the tumors take several months to develop, exhibit variable growth, and are detected only in a small fraction of these genetically engineered mice, limiting their use for therapeutic evaluation. Direct implantation of patient-derived meningioma tumor pieces into immunodeficient mice has been attempted, but intracranial sites of inoculation may be challenging, and the growth of benign tumors is limited [Andersen et al., J Transl Med. 2023; 21:764(2023 )]. Alternatively, primary meningioma cells can be prepared to generate xenografts, but these primary cultures have limited lifespan. Several grade-1meningioma cell lines have been generated by transforming primary tumor cell cultures with an oncogene or an oncogene plus telomerase; however, these oncogene-transformed cell lines exhibit altered growth signaling and behavior [Baia et al., J Neurooncol., 78:113-121 (2006)]. By using only telomerase, Püttmann et al. [Lab Invest., 85:1163-1171 (2005)] generated the Ben-Men-1 meningioma cell line from a sporadic grade-1 tumor. We showed that Ben-Men-1 carries an NF2 mutation and does not express the merlin protein [Burns et al., Cancer Res., 73:792-804 (2013)]. We also generated luciferase-expressing Ben-Men-1-LucB cells, which are capable of growing as intracranial xenografts in immunodeficient mice. These xenografts grow slowly along the skull base like a benign meningioma, and we have used this orthotopic NF2-deficient meningioma model to identify several potent targeted drugs, such as brigatinib. However, the Ben-Men-1 cell line was established from a sporadic grade 1 meningioma occurring in a 68-year-old patient and may not completely reflect the biology of NF2-SWN related meningiomas which often develop in younger patients. In addition to distinct molecular features, NF2-SWN-related meningiomas tend to exhibit more aggressive clinical behavior [Kirches et al., Acta Neuropathol., 142:873-886 (2021)]. Here we describe the generation of the first telomerase-immortalized NF2-SWN related meningioma cell line, AG-NF2-Men, from a grade 1 tumor of a young adult with NF2-SWN.

Like most pediatric tumors, the original tumor from which the AG-NF2-Men cell line was derived has low mutational burden (1 mutation/Mb). We identified mutations only on two genes, a splice site mutation in NF2 on chromosome 22 and a duplication of exons 2-3 in MYC on chromosome 8. FISH analysis confirmed the loss of one chromosome 22 in both the original tumor and immortalized AG-NF2-Men cells, suggesting the loss of heterozygosity in NF2. Consistently, we did not detect any wild-type merlin protein in AG-NF2-Men cells. Due to NF2 loss, the AG-NF2-Men cell line expressed several merlin-regulated RTKs, such as EGFR, ErbB3, and IGF-1R, and responded to their cognate ligands. Like the Ben-Men-1 cell line and other NF2-deficient meningiomas, activation of these RTKs leads to robust phosphorylation and activation of their downstream signaling molecules AKT and ERK1/2 to drive meningioma cell proliferation. Although the effect of duplication of MYC exons 2 and 3, which include the entire MYC protein coding sequence [Carabet et al., Int J Mol Sci., 20:120 (2019)], is not known, IHC analysis readily detected MYC labeling in the nucleus of AG-NF2-Men tumor cells. As expected, the immortalized AG-NF2-Men cells re-expressed abundant telomerase activity, while the original tumor cells showed negligible telomerase activity. In addition, the AG-NF2-Men cell line expressed several markers characteristic of meningiomas, such as desmoplakin, vimentin, and EMA. We have generated the luciferase-expressing AG-NF2-Men-Luc2 cells and found that these cells could grow steadily as intracranial, but not subcutaneous, xenografts, suggesting the importance of the tumor microenvironment in supporting meningioma growth.

Previously, we, as a member of the Synodos for NF2 consortium in collaboration with NCATS, identified brigatinib and INK128 with promising efficacies in the NF2-deficient Ben-Men-1 model [Chang et al., PLoS One, 16: e0252048 (2021)]. We now showed that like in the Ben-Men-1 model, brigatinib and INK128 exhibited similar growth-inhibitory and anti-tumor effects in the AG-NF2-Men model, such as comparable IC₅₀ concentrations, reduced phosphorylation of merlin-regulated RTKs, such as EGFR, ErbB3, and IGF-1R, and their downstream AKT and ERK1/2 kinases, as well as tumor shrinkage particularly in the first three weeks of treatment. Together with the INTUITT-NF2 clinical trial, which demonstrates the clinical activity of brigatinib in NF2-SWN patients with meningiomas [Plotkin et al, N Engl J Med., 390:2284-2294 (2024)], these results further support the idea of extending the evaluation of brigatinib to treat sporadic meningiomas with pathogenic variants in the NF2 gene.

As NF2-deficient tumors often exhibit deregulated mTOR signaling (James et al., 2009, 2012), mTOR inhibitors have been investigated as potential treatments, with several having in vitro and in vivo preclinical activity [Goutagny et al., J Neurooncol., 133:443-445 (2017)]. However, the first-generation rapamycin analogs, such as everolimus, show limited clinical efficacy, possibly due to incomplete inhibition of mTORC1 [Karajannis et al., Mol Cancer Ther., 20:1584-1591 (2021)]. Second-generation mTOR inhibitors, such as INK128 (sapanisertib) and AZD2014 (vistusertib), which directly block mTOR kinase activity, suppress both the mTORC1 and mTORC2 complexes. AZD2014 shows some clinical efficacy with stable disease in NF2 patients with progressive or symptomatic meningiomas, but unfortunately the dosing regimen (125 mg orally twice daily for two consecutive days each week) was poorly tolerated [Jordan et al., Neurooncol Adv., 5: vdad041 (2023)]. These results suggest a need of dose reduction or combining with other targeted agents. Third-generation mTOR inhibitors, such as RMC-6272 and RMC-5552, were developed to avoid the poor tolerability of mTOR kinase inhibitors while maintaining their high potency [Burnett et al., J Med Chem., 66:149-169 (2023)]. Bhattacharyya et al. [Neurooncol Adv., 6: vdae024 (2024)] recently reported that RMC-6272 elicits superior anti-proliferative activity with durable inhibition of 4E-BP1 phosphorylation in NF2-deficient meningioma cells, compared to first-and second-generation mTOR inhibitors. Also, we showed that RMC-6272 effectively blocks the growth of intracranial NF2-deficient meningioma xenografts.

Studies have shown that NF2 inactivation leads to dysregulation of multiple signaling pathways and that NF2-SWN tumors exhibit heterogeneity with variable responses to treatment, suggesting a need for combination treatments. Since brigatinib or INK128 alone, while exhibiting potent anti-tumor effects, did not eliminate NF2-deficient meningiomas [Angus et al., Neuro Oncol., 20:1185-1196 (2018)], we found that their combination elicited growth suppressive synergy in both Ben-Men-1 and AG-NF2-Men cells. Mechanistically, the brigatinib+INK128 combination produced a stronger suppression of p-AKT signaling and p-4EBP1 compared to the individual drugs. The abrogation of p-4EBP1 is particularly interesting as the ability to decrease p-4EBP1 correlates with clinical efficacy [Burnett et al., J Med Chem., 66:149-169 (2023)]. We also found that INK128 likely induces feedback compensatory phosphorylation of p-AKT(T308) and p-ERK. Activated p-AKT and mTOR typically engage negative feedback inhibition pathways to prevent their unrestrained growth signaling, and drugs that interfere with this regulation and can paradoxically promote mitogenic growth through compensatory activation of AKT and ERK1/2 [Rodrik-Outmezguine et al., Cancer Discov., 1:248-259 (2011)]. Thus, it is reassuring that the brigatinib+INK 128 combination does not display re-activation of these molecules.

Transcriptomic analyses of AG-NF2-Men cells treated for 24-hours with brigatinib, INK128, or their combination revealed that brigatinib or INK128 alone affected the expression of only a limited number of genes. However, the brigatinib+INK128 combination-treated cells displayed a startlingly shift in their transcriptome, with over 5000 DEGs. Pathway analysis showed that several of these DEGs correlated with the inhibition of the EGFR/ErbB3 and AKT pathways, consistent with our findings from Western analysis of treated cells. Surprisingly, several YAP-regulated genes were suppressed by the brigatinib+INK128 combination. Since merlin negatively regulates the Hippo/YAP signaling [Striedinger et al., Neoplasia, 10:1204-1212 (2008)], these results suggest that the brigatinib+INK128 combination broadly suppresses various mitogenic pathways deregulated in meningioma cells with merlin loss. It will be interesting to see whether the brigatinib+INK128 combination inhibits YAP transcriptional activity, and, if so, the mechanisms by which it occurs.

Importantly, the brigatinib+INK128 combination showed enhanced tumor suppression of intracranial AG-NF2-Men-Luc2 xenografts and was well-tolerated overall as treated mice did not show overt weight loss. When the AG-NF2-Men-Luc2 tumors were re-treated after temporary drug cessation, we found that the combination-treated tumors took longer before they resumed growth and that tumor regression promptly occurred upon re-administration of the combined regimens. In contrast, single-agent INK128 showed transient tumor regression during the first week of re-treatment before resuming growth, suggesting the treated tumor had developed partial resistance to INK128.

Overall, we have described and characterized the AG-NF2-Men cell line as the first NF2-SWN related meningioma cell line generated exclusively through telomerase immortalization. We also generated an orthotopic, quantifiable, NF2-SWN related meningioma model using luciferase-expressing AG-NF2-Men-Luc2 cells. In addition to the Ben-Men-1-LucB model that we established previously, this model should serve as a valuable addition to identify novel drugs and drug combinations to treat NF2-SWN related and NF2-deficient sporadic meningiomas. Since the third-generation mTOR inhibitor RMC-5552 shows potent preclinical activity in several cancer models and demonstrates favorable tolerability in clinical trial, the enhanced anti-tumor effects of the brigatinib+INK128 combination that we observed suggest that combining brigatinib with an mTOR inhibitor may be a promising treatment to improve the clinical care of NF2-SWN patients with meningiomas.

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, while various theories are presented describing possible mechanisms through with the compounds are effective, the compounds are effective regardless of the particular mechanism employed and the inventors are therefore not bound by theories described herein. The invention is not limited to the exact details shown and described, for variations obvious to one skilled in the art will be included within the invention defined by the claims.

What is claimed is:

1. A method of generating a tumor cell line, comprising immortalizing meningioma cells obtained from a benign tumor by introducing telomerase reverse transcriptase to provide telomerase immortalized meningioma cells, and passaging the telomerase immortalized meningioma cells in growth medium together with untransformed meningioma cells until the untransformed cells undergo senescence to provide a tumor cell line comprising telomerase-immortalized meningioma cells.

2. The method of claim 1, wherein the meningioma cells are NF2-deficient.

3. The method of claim 1, wherein the meningioma cells are human meningioma cells.

4. The method of claim 3, wherein the cells are obtained from a subject having neurofibromatosis type 2-related schwannomatosis (NF2-SWN).

5. A tumor cell line comprising telomerase-immortalized meningioma cells obtained from a benign tumor.

6. The tumor cell line of claim 5, wherein the tumor cell line is prepared by the method of claim 1.

7. The tumor cell line of claim 5, wherein the meningioma cells are NF2-deficient.

8. The tumor cell line of claim 5, wherein the meningioma cells are human meningioma cells.

9. The tumor cell line of claim 8, wherein the cells are obtained from a subject having NF2-SWN.

10. An animal model of NF2-deficient meningioma, comprising a mammal implanted with telomerase-immortalized, luciferase-expressing meningioma cells obtained from a benign tumor.

11. The animal model of claim 10, wherein the telomerase-immortalized meningioma cells are human telomerase-immortalized meningioma cells.

12. The animal model of claim 10, wherein the telomerase-immortalized meningioma cells are prepared by transfecting meningioma cells obtained from a benign tumor with telomeres reverse transcriptase to provide telomerase immortalized meningioma cells and passaging the telomerase immortalized meningioma cells in growth medium together with untransformed meningioma cells until the untransformed cells undergo senescence.

13. The animal model of claim 10, wherein the mammal is a mouse.

14. The animal model of claim 13, wherein the telomerase-immortalized meningioma cells are implanted at the base of the skull of the mouse.

15. A method of treating meningioma in a subject in need thereof by administering a therapeutically effective amount of brigatinib to the subject.

16. The method of claim 15, wherein the meningioma is NF2-deficient meningioma.

17. The method of claim 15, wherein an mTOR inhibitor is also administered to the subject.

18. The method of claim 17, wherein the mTOR inhibitor is INK128.

19. The method of claim 15, wherein the subject is human.
